SEQUENCE LISTING (5 sequences)

SEQ ID NO: 1 - protein, 343 aa, Homo sapiens
SEETKENEGFTVTAEGKGGGTLSVVTMYHAKAKDQLTCNKFDLKVTIKPAPETEKRPDEAKNTMILEICTRYRGNQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 2 - protein, 410 aa, Homo sapiens
YVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMGFSTWDNDNDKFEGNCAEGDGSGWWMNKCHAGHLNGVYYQGGTYSKASTPNGYDNGIIWATKTRWYSMKKTTMKIIPFNRLTIGEGGQHHLGGAKQAGDV

SEQ ID NO: 3 - protein, 201 aa, Homo sapiens
MALSWVLTVPEPPTSAGSPDPIVCQPSTGAHHNATLDQITGKWFYIASAFRNEEYNKSVQEIQATFFYFTPNKTEDTIFLREYQTRQDQCIYNTTYLNVQRENGTISRYVGGGEHFAHLLILRDTKTYMLAFDVNDEKNWGLSVYADKPETTKEGLGEFYEALDCLRIPKSDVVYTDWKKDKCEPLEKQHEKERKQEEGES

SEQ ID NO: 4 - protein, 406 aa, Homo sapiens
MSALGAVIALLLWGQLFAVDSGNDVTDIADDGCPKPPEIAHGYVEHSVRYQCKNYYKLRTEGDGVYTLNDKKGWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSDVDIGLIKLKQKVSVNERVMPICLPSKDYAEVGRVGYVSGWGRNANFKFTDHLKYVMLPVADQDQCIRHYEGSTVPEKKTPKSPVGVGPILNEHTFCAGMSKYQEDTCYGDAGSAFAVHDLEEDTWYATGILSFDKSCAVAEYGVYVKVTSIQDWVQKTIAEN

SEQ ID NO: 5 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

(19) United States
(12) Patent Application Publication
(10) Pub. No.: US 2011/0091991 A1
(43) Pub. Date: Apr. 21, 2011

(54) Protein Biomarkers for Alzheimer's Disease Detection

(75) Inventors: Shiau-Shiun Guan, Taoyuan City (TW); Shui-Cheng Lee, Longtan Township (TW); Chun-Chia Cheng, Yonghe City (TW)

(73) Assignee: ATOMIC ENERGY COUNCIL-INSTITUTE OF NUCLEAR ENERGY RESEARCH, Taoyuan (TW)

(21) Appl. No.: 12/603,522

(22) Filed: Oct. 21, 2009

(51) Int. Cl. G01N 33/53

(52) U.S. Cl. 436/501

(57) ABSTRACT

Protein biomarkers are selected for diagnosing Alzheimer's disease. Samples of Alzheimer's disease are used to find the biomarkers. It is done through methods including 2-dimentional differential in-gel electrophoresis (2D-DIGE), isotope-coded protein labeling (ICPL) and western blotting. Through examining density differences of the selected biomarkers, Alzheimer's disease can be early diagnosed or prevented.

### FIELD OF THE INVENTION

The present invention relates to detecting Alzheimer's disease (AD); more particularly, relates to obtaining protein biomarkers and obtaining a detection tool with the protein biomarkers to detect AD by examining density differences of the protein biomarkers in serum.

### DESCRIPTION OF THE RELATED ARTS

AD is often found in old men of age above 65, which may cause cognitive dysfunctions. Some areas in the brain of an infected man are affected by AD, like basal forebrain and hippocampus. The diagnosis and therapy of AD cost high while drugs for AD can only make condition better but not stop the disease. Hence, early diagnosis is the only hope.

However, until now, there are still neither totally effective examination methods for diagnosing AD nor totally effective drugs for curing AD. Cognex and Aricept are two drugs for curing AD. They are acetylcholine cholinesterase inhibitors. By blocking acetylcholine cholinesterase, biolysis of acetylcholine is inhibited with the amount of acetylcholine in brain raised. Yet, the two drugs still do not cure AD.

On examining the brains of the AD-affected men, not only neuron and synapse are found reduced in brain cortex, but also protein deposits are found outside of the neuron, which phenomenons are closely related to AD. Until now, the only way to explicitly diagnose AD is to find plaques and tangles in brain. Hence, non-invasive protein biomarkers are the best choice for diagnosing AD since no tissue section observation is possible for a living body.

Protein biomarkers are usually obtained from body fluids, like serum, saliva, cerebrospinal fluid, etc. However, the body fluids may flow over the whole body and their relationships to the disease is hard to be confirmed. Besides, proteins in some cancer patients' serums have increased expressions, which may mean cancer cells may requires more factors for proliferation. This is why later-stage cancer is easier to be found, which is not available for diagnosing early-stage cancer.

Hence, the prior arts do not fulfill all users' requests on actual use.

### SUMMARY OF THE INVENTION

The main purpose of the present invention is to obtain protein biomarkers and a detection tool with the protein biomarkers to detect AD by examining density differences of the protein biomarkers in serum.

To achieve the above purpose, the present invention is protein biomarkers for AD detection, comprising five protein biomarkers, where the first protein biomarker has a protein identity of fibrinogen γ′, a molecular weight of 46.3 kDa and an isoelectric point of 5.54; the second protein biomarker has a protein identity of complement component 3, a molecular weight of 39.5 kDa and a isoelectric point of 4.79; the third protein biomarker has a protein identity of α-1 acid glycoprotein, a molecular weight of 23.5 kDa and a isoelectric point of 4.79; the fourth protein biomarker has a protein identity of haptoglobin, a molecular weight of 45.2 kDa and a isoelectric point of 6.13; and the fifth protein biomarker has a protein identity of transthyretin, a molecular weight of 15.9 kDa and a isoelectric point of 5.43. Accordingly, novel protein biomarkers for AD detection are obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the view showing the flow of detecting the protein biomarkers according to the preferred embodiment of the present invention;

FIG. 2 is the view showing the arrangement of the serum samples for 2D-DIDE;

FIG. 3 is the view showing the first 2D-DIGE comparison image;

FIG. 4 is the view showing the second 2D-DIGE comparison image;

FIG. 5 is the view showing the identification of fibrinogen γ′;

FIG. 6 is the view showing the identification of complement component 3;

FIG. 7 is the view showing the identifications through 2D-DIGE;

FIG. 8 is the view showing the first identification through ICPL;

FIG. 9 is the view showing the second identification through ICPL;

FIG. 10 is the view showing the identifications through ICPL;

FIG. 11 is the view showing the identification of fibrinogen γ′ through western blotting;

FIG. 12 is the view showing the identification of complement component 3 through western blotting;

FIG. 13 is the view showing the identification of α-1 acid glycoprotein through western blotting;

FIG. 14 is the view showing the identification of haptoglobin through western blotting; and

FIG. 15 is the view showing the identification of transthyretin through western blotting.

### DESCRIPTION OF THE PREFERRED EMBODIMENT

The following description of the preferred embodiment is provided to understand the features and the structures of the present invention.

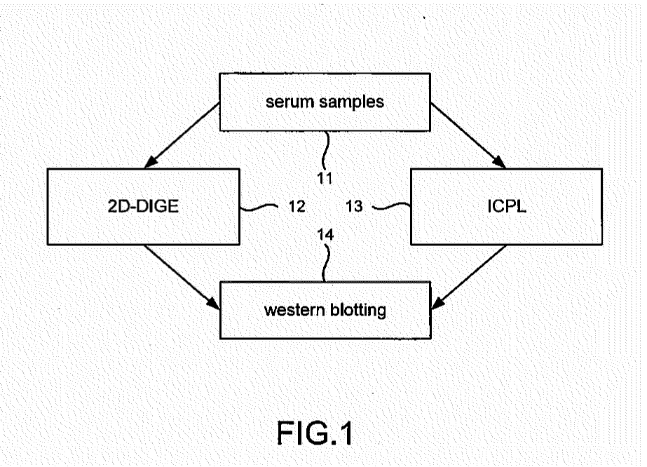

Please refer to FIG. 1, which is a view showing a flow of detecting protein biomarkers according to a preferred embodiment of the present invention. As shown in the figure, the present invention is protein biomarkers for Alzheimer's disease (AD) detection. The protein biomarkers are obtained through the following steps:

(a) Obtaining serum samples 11: A plurality of AD serum samples and a plurality of normal serum samples are obtained.

(b) Obtaining protein identity: Protein identities of AD protein biomarkers are obtained through the following two methods:

(b1) 2D-DIGE 12: 2-dimensional differential in-gel electrophoresis (2D-DIGE) is used. Fluorescein dye (CyDye) is directly applied on the two kinds of serum samples put on a film. The serum samples are processed through analyses of isoelectric focusing (IEF) and SDS polyacrylamide gel electrophoresis (SDS-PAGE) to separate proteins. Then AD protein biomarkers are figured out by an image analysis tool (Decyder 6.5) for obtaining their protein identities through a mass spectroscope.

(b2) ICPL 13: Isotope-coded protein labeling (ICPL) is used to label proteins having different mass weights in the two kinds of serum samples. A mass spectroscope is used to analyze differences between the two kinds of serum samples. Then peptides fragments having differences between the two kinds of serum samples (i.e. AD protein biomarkers) are automatically selected according to fore-settings; and their protein identities are obtained by a mass spectroscope.

(c) Confirming protein identities 14: At last, the protein biomarkers obtained in step (b1) and step (b2) are processed through western blotting to confirm their protein identities.

The selected AD protein biomarkers are biomarkers related to hemopoietic function, immunoreaction, apoptosis and neuron supply, which are fibrinogen γ′, complement component 3, α-1 acid glycoprotein, haptoglobin and transthyretin. The selected protein biomarkers are fixed on protein biochips to diagnose AD by examining differences between the protein biomarkers in the serum samples.

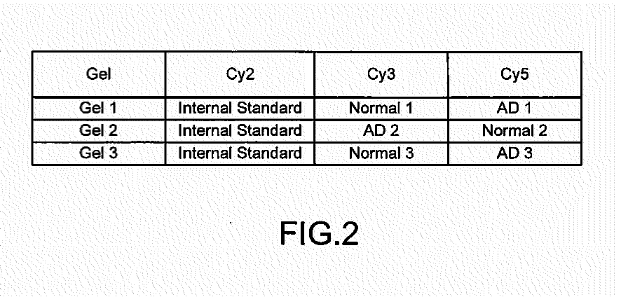

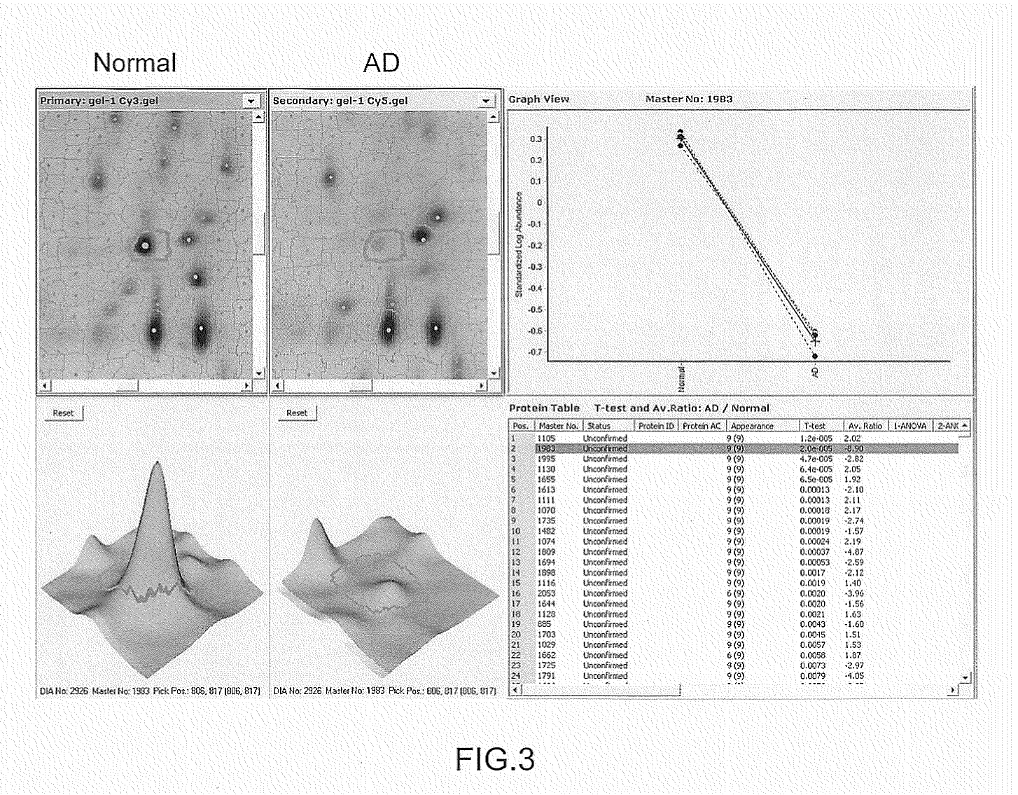

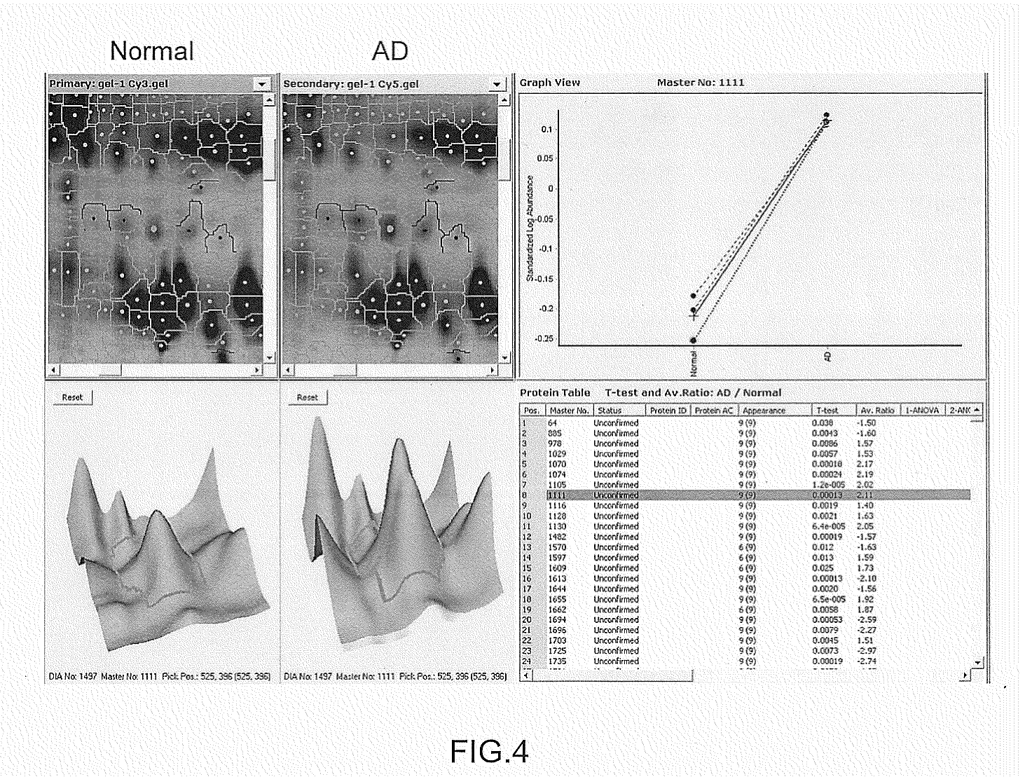

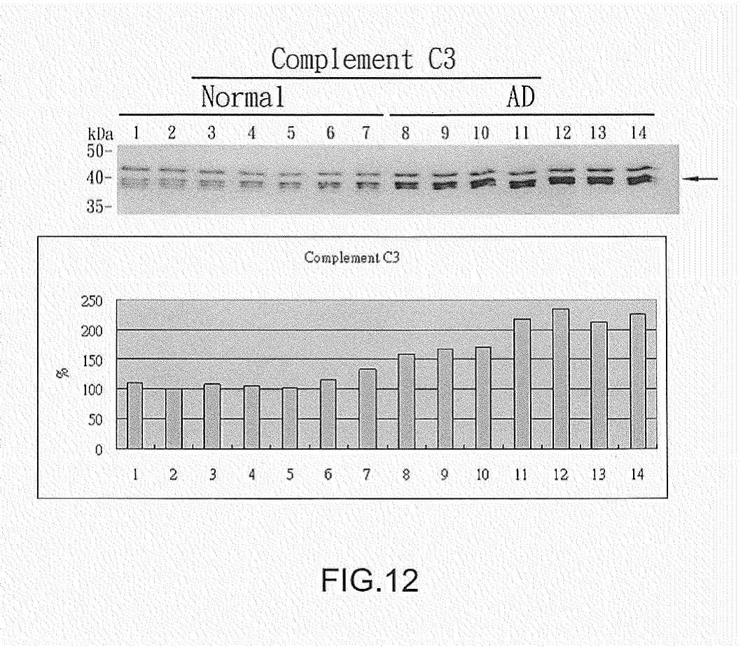

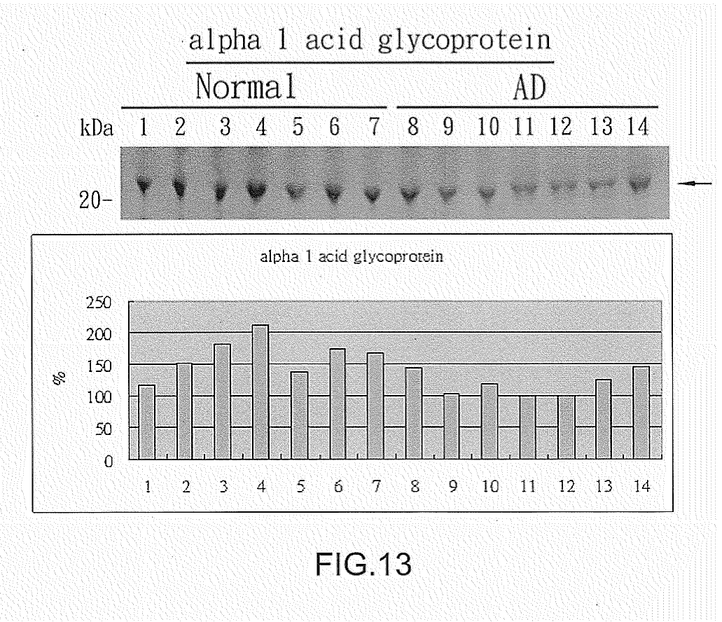

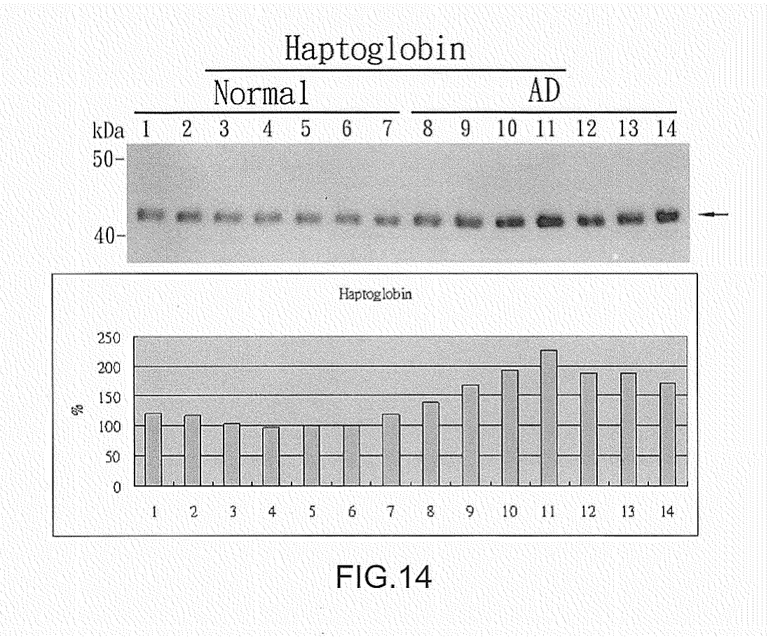

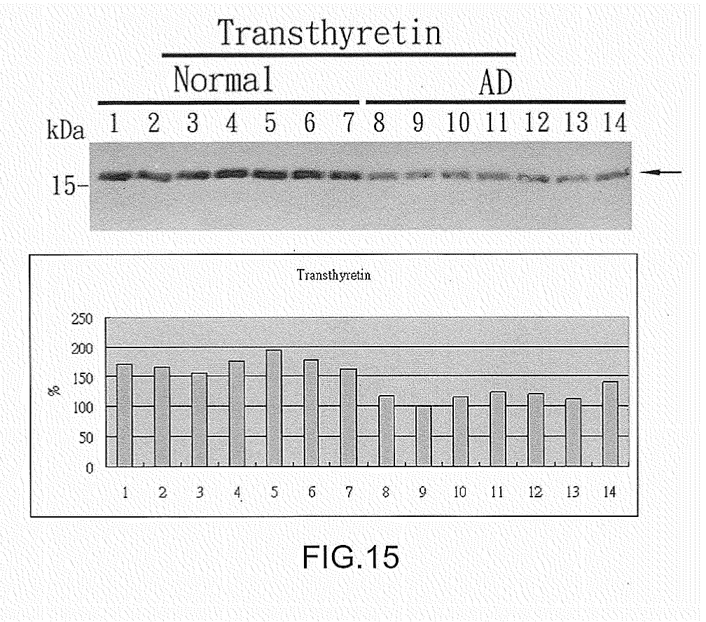

Please refer to FIG. 2 to FIG. 15, which are a view showing arrangement of serum samples for 2D-DIDE; views showing a first and a second 2D-DIGE comparison images; views showing identification of fibrinogen γ′ and complement component 3; a view showing identifications through 2D-DIGE; views showing a first and a second identifications through ICPL; a view showing identifications through ICPL; and views showing identifications of fibrinogen γ′, complement component 3, α-1 acid glycoprotein, haptoglobin and transthyretin through western blotting. As shown in the figures, the present invention selects AD protein biomarkers, comprising fibrinogen γ′, complement component 3, α-1 acid glycoprotein, haptoglobin and transthyretin.

### [State 1] Serum Samples Collection

15 AD serum samples and 20 normal serum samples are collected to obtain a total of 35 serum samples.

### [State 2] 2D-DIGE

### i) Flow of 2D-DIGE

The serum samples are divided into two groups. 3 serum samples are obtained from each group; and 6 serum samples are thus put on 3 films. Fluorescein dye of Cy2 is used to label mixed serum samples of the normal serum samples for obtaining internal standards. Then, as shown in FIG. 2, the 6 serum samples are labeled with Cy3 and Cy5. Therein, the serum samples of the same sort are labeled with Cy3 and Cy5 separately to avoid fluorescence interference in between. Then the separately labeled serum samples are analyzed together on three films.

Because isoelectric points and molecular weights of the serum samples are different, IEF and SDS-PAGE are according to their electric charges and molecular sieve effects.

### ii) Imaging and Analyzing

A luminescence scanner is used to obtain images of the films after electrophoresis. Then the images labeled with Cy3 and Cy5 are put together for coloring different proteins with different colors, where Cy3 is green and Cy5 is red and yellow means the same protein expression. Thus, different protein expressions are obtained through different colors.

Then 9 images of the 3 films are inputted to be analyzed by DeCyder 6.5. The Cy2 images are set as internal standards and the other images of normal serum samples and AD serum samples are arranged accordingly to obtain 3 sets of images. Then the 9 images are processed through differential in-gel analysis (DIA), where proteins having more than 10000 spots are labeled. Later, only proteins having spots greater than 60000 are shown to eliminate background noises; and, thus, ratios of Cy3 images or Cy5 images to the Cy2 images (the internal standard) are obtained for comparisons and analyses between the films with less human errors and more reliable results.

The next step is biological variation analysis. At first, correctness of locations of the labeled proteins is checked, which is manually done to correct wrongly labeled proteins and to correspond proteins in the 3 films to internal standards. Then, after correcting the wrongly labeled proteins, statistic analysis is processed to obtain T-test values and one-way variance analysis values (P values) for the proteins.

iii) Possible AD Protein Biomarkers

According to the T-test values and the P values, proteins whose reliability is higher than 95% (a value smaller than 0.05) are selected. Two AD protein biomarkers are thus obtained: a first AD protein biomarker are selected from the 9 images, whose T-test value is 2×10⁻⁵ and is a down regulated protein; and a second AD protein biomarker is selected from the 9 images, whose T-test value is 1.3×10⁻⁴ and is an up regulated protein

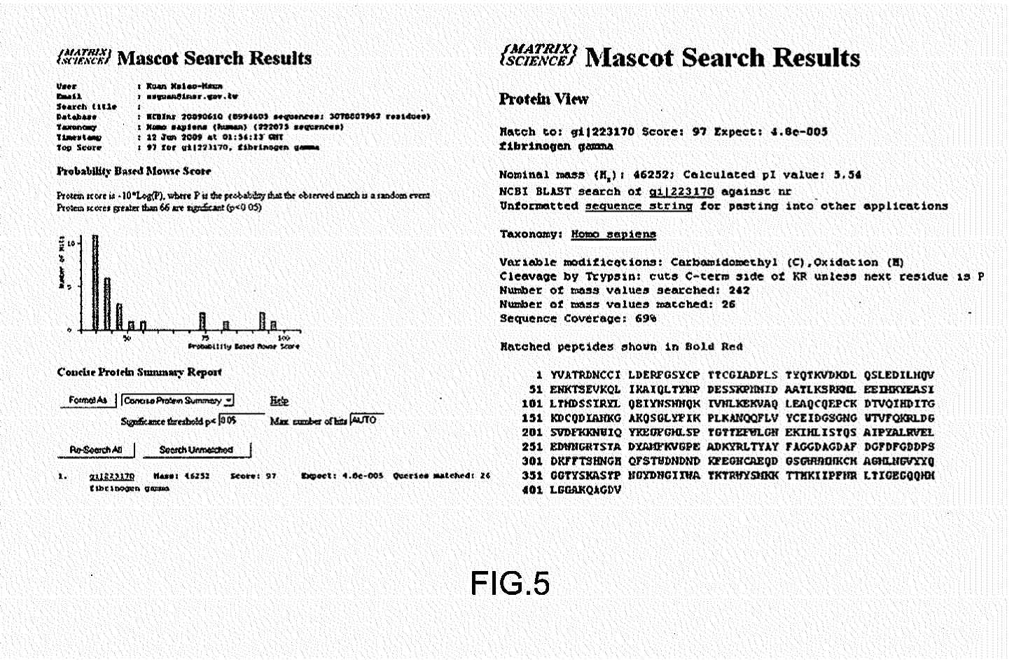

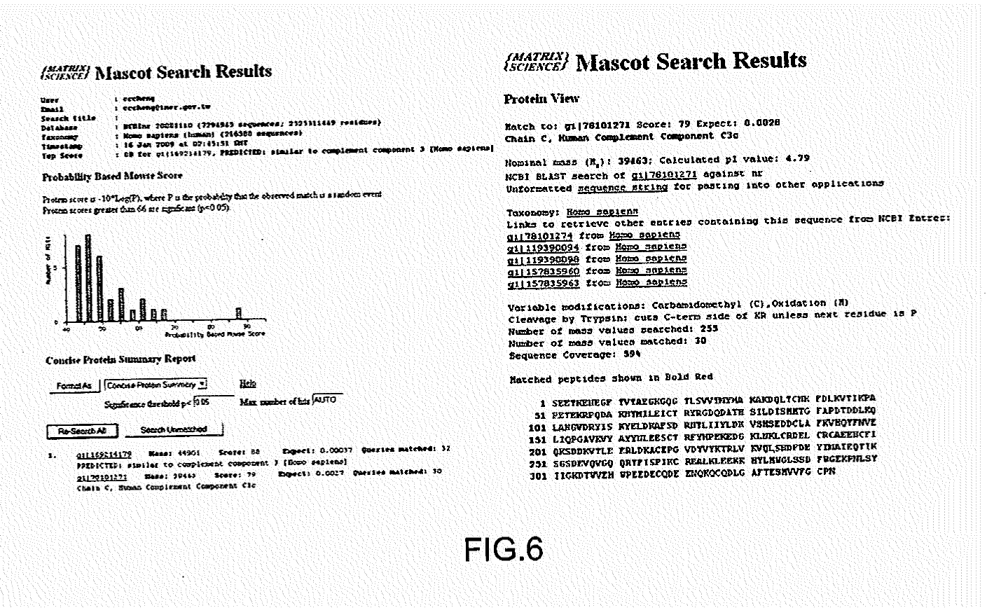

After 2D-DIGE, locations of the two AD protein biomarkers are marked. After fluorescent staining, the proteins are released from the films by UV light. Then trypsin is used for hydrolysis to analyzed protein spots through a mass spectroscope (MALDI-TOF/TOF). Then peptide molecular weights are obtained through a data analysis tool (FlexAnalysis) and a biological analysis tool (BioTool). In FIG. 5 and FIG. 6, the data are transferred to a MASCOT protein identity workstation to be analyzed and compared with NCBI protein database.

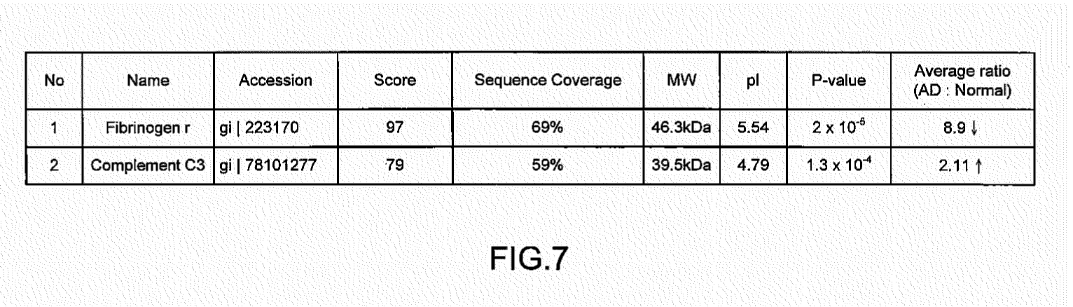

In FIG. 7, the first AD protein biomarker has a reliability of 97 and its protein identity is fibrinogen γ′. Fibrinogen γ′ has a molecular weight of 46.3 kDa and an isoelectric point of 5.54; and its sequence overlapping rate to the protein biomarker in human serum is at least 69%. The second AD protein biomarker has a reliability of and its protein identity is complement component 3. Complement component 3 has a molecular weight of 39.5 kDa and an isoelectric point of 4.79; and its sequence overlapping rate to the protein biomarker in human serum is at least 59%.

Thereafter, through using enzyme linked immunosorbent assay (ELISA) or protein suspension array, the protein biomarkers in serum are confirmed by differences are obtained through absolute quantification.

### [State 3] ICPL

### Hyper 3.2 Analysis Results

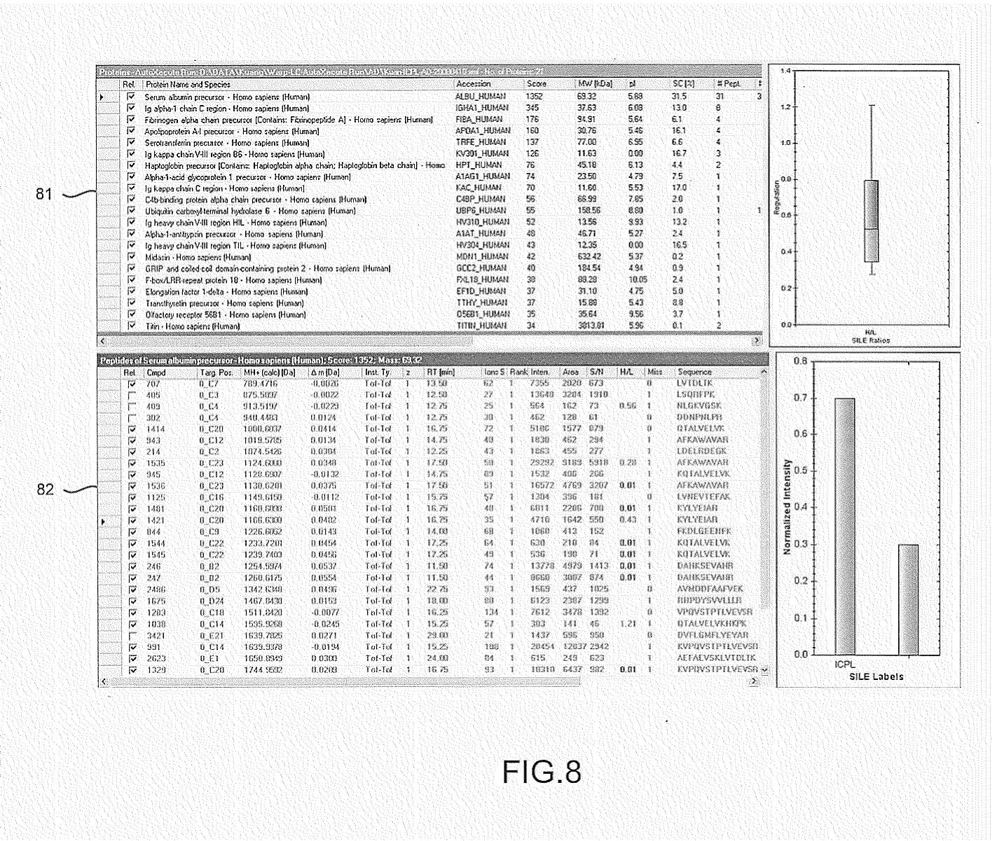

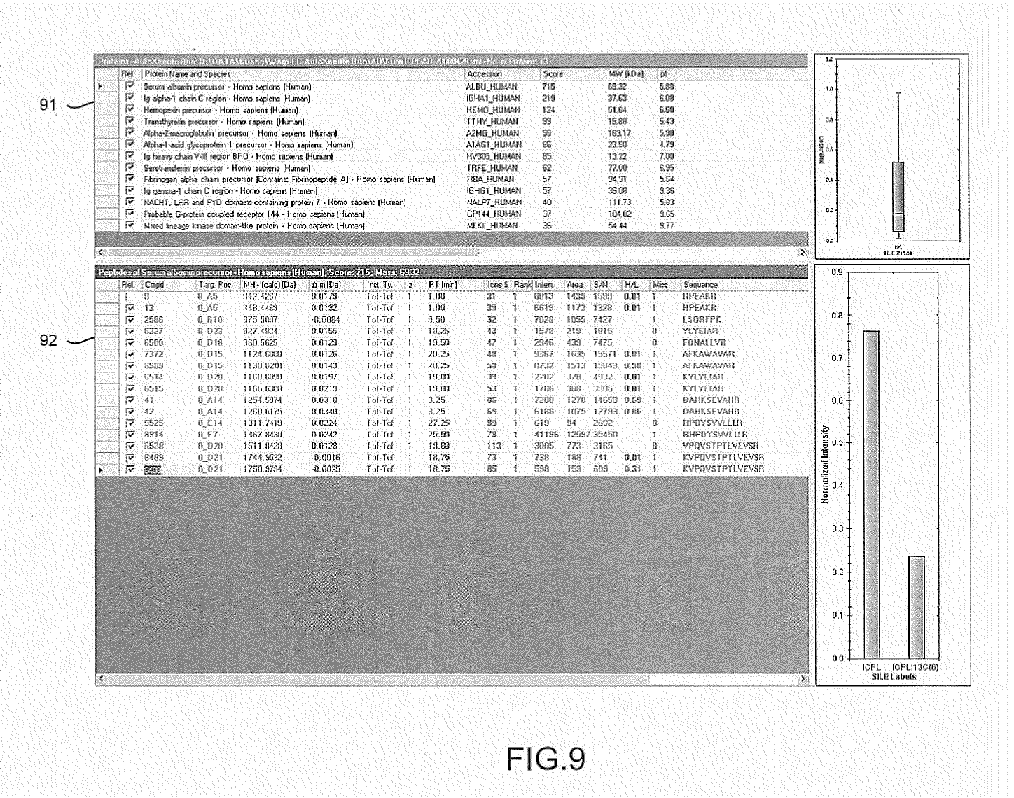

¹²C and ¹³C are used as markers to label the serum samples, which are labeled on lysine lateral chain and free amino group. By using Hyper 3.2 through a protein or peptide separation system, a mass spectroscope (MALDI-TOF/TOF) is sued for analysis. In FIG. 8 and FIG. 9, the upper part 81, 91 shows data of proteins, including name, molecular weight, isoelectric point and identified peptide number; and the lower part 82, 92 shows data of corresponding peptides fragments to the proteins, including elution time for chromatography, position on MALDI dish and identified amino acid sequence. Thus, differences between labeled proteins are obtained.

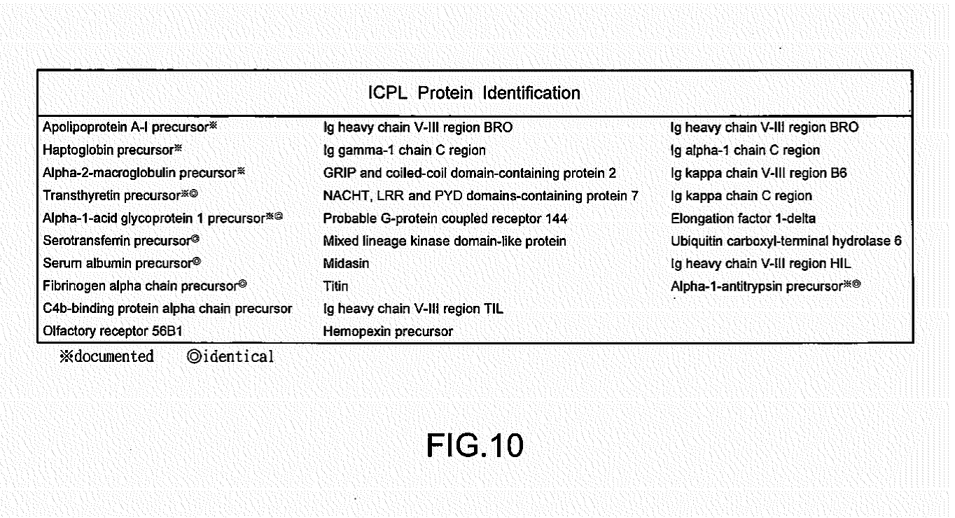

In FIG. 10, after two analyses, a few proteins are identical, including serum albumin precursor, transthyretin precursor, α-1-acid glycoprotein 1 precursor, serotransferrin precursor and fibrinogen α chain precursor, with some other proteins documented. In the first analysis, MALDI samples are analyzed through MS and are fractionated through HPLC to find signals having wave pitches higher than 800 and molecular weight intervals greater than 6 Da. In the second analysis, identified proteins are those that have differences between expression of the AD serum samples and that of the normal serum samples.

### [State 4] Western Blotting

### i) Flow of Western Blotting

14 serum samples are divided into two groups, each having 7 samples. After quantification, each sample is added with a buffer having a two-times size to that of the sample. Then the samples are heated for 10 minutes at 100° C. for protein denaturation.

Because molecular weights of the serum samples are different, SDS-PAGE is processed to separate proteins according to their molecular sieve effects.

Then, through western blotting, proteins in the films are transferred to protein absorption holes on a PVDF transfer film while the other holes are filled with skimmed milk. And then five proteins of primary antibodies, which are fibrinogen γ′, complement component 3, α-1 acid glycoprotein, transthyretin and haptoglobin, are reacted with secondary antibodies for identification.

### ii) Results and Statistics Obtained Through a Luminescence Analysis Tool

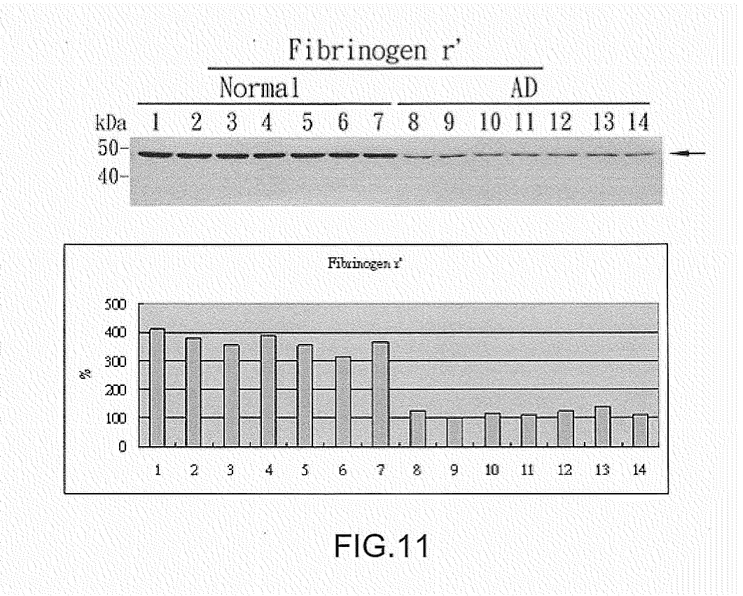

In FIG. 11 to FIG. 15, the reacted PVDF transfer film is processed through luminescence development with a coloring agent (Enhanced ChemiLuciferase) to be examined by a luminescence analysis tool (LAS-4000mini). Then, by using an image quantification analysis tool for western blotting (Multi Vauge V3.2), signals of the developed images are processed for quantification analysis to obtain a bar graph for comparison.

After identification through western blotting, differences of proteins between the normal serum samples and the disease serum samples are clearly shown with the luminescence analysis tool, where the different protein biomarkers comprises fibrinogen γ′, complement component 3, α-1 acid glycoprotein, haptoglobin and transthyretin; and are identical to those obtained by the proteomic analyses tools (2D-DIGE and ICPL).

Thus, protein biomarkers are selected from AD serum samples to be analyzed by proteomic analyses tools for finding differential biomarkers. Then western blotting is used to identify the protein biomarkers, which is used to obtain a detection tool. The detection tool is used for detecting AD by examining existences of the protein biomarkers in serum. And the detection tool thus obtained has high accuracy and sensitivity, which is fit for early diagnosis and early prevention.

To sum up, the present invention is protein biomarkers for AD detection, where protein biomarkers are selected from AD serum samples to be analyzed by proteomic analyses tools for finding differential biomarkers; western blotting is used to identify the protein biomarkers for making a detection tool with the protein biomarkers; the detection tool is used for detecting AD by examining existences of the protein biomarkers in serum; and the detection tool thus obtained has high accuracy and sensitivity and is fit for early diagnosis and early prevention.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

1. Protein biomarkers for Alzheimer's disease (AD) detection, comprising:
a first protein biomarker, said first protein biomarker having a protein identity of fibrinogen γ′ (SEQ ID NO:2), a molecular weight of 46.3 kDa and an isoelectric point of 5.54;
a second protein biomarker, said second protein biomarker having a protein identity of complement component 3 (SEQ ID NO:1), a molecular weight of 39.5 kDa and an isoelectric point of 4.79;
a third protein biomarker, said third protein biomarker having a protein identity of a-1 acid glycoprotein (SEQ ID NO:3), a molecular weight of 23.4 kDa and an isoelectric point of 4.79;
a fourth protein biomarker, said fourth protein biomarker having a protein identity of haptoglobin (SEQ ID NO:4), a molecular weight of 45.2 kDa and an isoelectric point of 6.13; and
a fifth biomarker, said fifth protein biomarker having a protein identity of transthyretin (SEQ ID NO:5), a molecular weight of 15.9 kDa and an isoelectric point of 5.43.

2. The biomarkers according to claim 1, wherein said protein biomarkers are obtained through the following steps of: (a) obtaining serum samples of AD; (b) finding differential protein biomarkers through proteomic analyses; and (c) confirming said protein biomarkers through western blotting.

3. The biomarkers according to claim 1, wherein said proteomic analyses comprises 2-dimensional differential in-gel electrophoresis (2D-DIGE) and isotope-coded protein labeling (ICPL).

4. The biomarkers according to claim 1, wherein a detection tool is obtained with said protein biomarkers; and wherein AD is detected by said detection tool through examining density differences of said protein biomarkers in serum.

5. The biomarkers according to claim 1, wherein said protein biomarkers are fixed on protein biochips.

6. A method for detecting Alzheimer's disease (AD) in a patient, comprising:
(a) obtaining serum samples from a patient;
(b) finding differential protein biomarkers in the serum through proteomic analyses; and (c) confirming said protein biomarkers through western blotting, wherein the protein biomarkers are those of claim 1.
